(19) United States
(12) Patent Application Publication
(10) Pub. No.: US 2001/0047197 A1
(43) Pub. Date: Nov. 29, 2001

(54) Methods and devices for in situ formation of vascular structures suitable for use as blood vessels

(76) Inventor: Mark J. Foley, Atherton, CA (US)

Correspondence Address: Carol LaSalle, Bozicevic, Field and Francis LLP, Suite 200, 200 Middlefield Road, Menlo Park, CA, 94025, US

(21) Appl. No.: 09/863,198

(22) Filed: May 22, 2001

Related U.S. Application Data
(60) Provisional application No. 60/206,439, filed on May 22, 2000.

(51) Int. Cl. A61F 2/06

(52) U.S. Cl. 623/1.12; 623/1.23

(57) ABSTRACT

Methods for forming in situ a vascular graft having a lumen adapted to place at least two blood vessels in fluid communication. A mandrel sized and configured to substantially define a lumen of a vascular graft is placed adjacent tissue located in a patient's body, and is substantially maintained in a desired position to form a vascular graft at the desired position. The vascular graft is then used to place at least two blood vessels in fluid communication with each other, for example, it is used as coronary artery bypass graft vessel to deliver blood from the aorta to the distal side of an occluded or partially occluded coronary artery. The invention also provides a system for forming a vascular structure having a lumen. The system includes a mandrel having an exterior surface sized and configured to form a vascular structure as a result of tissue healing around the surface of the mandrel, and a mechanism by which the mandrel is positioned in a desired location relative to tissue so that a vascular structure may form at the position. The mandrel is configured to allow the vascular structure to be used as a graft without harvesting or removing the vascular structure after its formation, for example; the mandrel may be removable or bioabsorbable.

### CROSS REFERENCE TO RELATED APPLICATIONS

Pursuant to 35 U.S.C. §119(e), this application claims priority to the filing date of U.S. Provisional Patent Application Serial No. 60/206,439 filed May 22, 2000, the disclosure of which is herein incorporated by reference.

### FIELD OF THE INVENTION

The invention relates generally to methods and devices for forming vascular structures in a patient's body, and more particularly methods and devices for forming such structures in situ.

### BACKGROUND OF THE INVENTION

A particular problem associated with treating vascular disease is the shortage of suitable grafts that may be used as bypass conduits. The grafts typically used in a coronary bypass procedure are native blood vessels, such as the internal mammary arteries (which may be used without harvesting) or the saphenous vein (which is harvested from the patient's leg). There is, however, in most patients a limited number of available suitable conduits. This problem is even more significant when treating patients with peripheral vascular disease.

Synthetic vascular graft materials have been under development for a very long time, yet to date their success has been essentially limited to use in large vessel applications, for example, treating abdominal aortic aneurysms. These materials, such as ePTFE and DACRON, have not fared as well treating smaller vessels, for example, occluded or partially occluded coronary arteries. Accordingly, those in the art continue search for improved ways of addressing the shortage of bypass conduits that exists in many patients.

### SUMMARY OF THE INVENTION

In one embodiment the invention provides a method for forming in situ a vascular graft having a lumen adapted to place at least two blood vessels in fluid communication. The method is carried out by providing a member sized and configured to substantially define a lumen of a vascular graft, placing at least a portion of the member adjacent tissue located in a patient's body, substantially maintaining the portion of the member in a desired position to form a vascular graft with a lumen at the desired position, and using the vascular graft to place at least two blood vessels in fluid communication without substantially moving the graft from the desired position.

In another embodiment the invention provides a method for forming a vascular structure in situ at a location in a patient's chest or thoracic cavity, the vascular structure having a lumen. The method is performed by providing a member adapted to define at least in part a vascular structure, placing at least a portion of the member in a desired position adjacent tissue located in a patient's chest or thoracic cavity, substantially maintaining the portion of the member in the desired position within the chest or thoracic cavity to form a vascular structure with a lumen at the desired position, and using the vascular structure to deliver blood.

In another embodiment the invention provides a system for forming a vascular structure having a lumen. The system includes a mandrel having an exterior surface sized and configured to form a vascular structure as a result of tissue healing around the surface of the mandrel, a positioning mechanism allowing the mandrel to be placed in a desired position relative to tissue so that a vascular structure may form at the position. The mandrel is configured to allow the vascular structure to be used as a graft without harvesting or removing the vascular structure after its formation, for example; the mandrel may be removable or bioabsorbable.

### BRIEF DESCRIPTION OF THE DRAWING FIGS.

FIGS. 1-3 are sequential perspective views schematically showing the creation of a vascular structure according to one embodiment of the invention;

FIGS. 4A and 4B are sectional views showing one end of the vascular formed according to FIGS. 1-3 with and without, respectively, a mandrel used to create the structure;

FIGS. 5A and 5B are sectional views showing the other end of the vascular formed according to FIGS. 1-3 with and without, respectively, the mandrel;

FIGS. 6A-6D are longitudinal sectional views sequentially showing formation of a vascular structure according to another embodiment of the invention wherein a vessel support is used to maintain the lumen of the vascular structure open;

FIGS. 7A-7C are perspective views of various devices for forming vascular structures according to other embodiments of the invention;

FIGS. 8-10 are perspective views sequentially showing formation of a vascular structure according to yet another embodiment of the invention;

FIGS. 11A-11D are photographs of vascular structures formed in animal studies carried out according to the present invention; and

FIG. 12 is a photograph of an end of one of the vascular structures shown in FIGS. 11A-11D.

### DETAILED DESCRIPTION OF THE DRAWING FIGS.

Before the present formulations and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more steps and equivalents thereof known to those skilled in the art, and so forth.

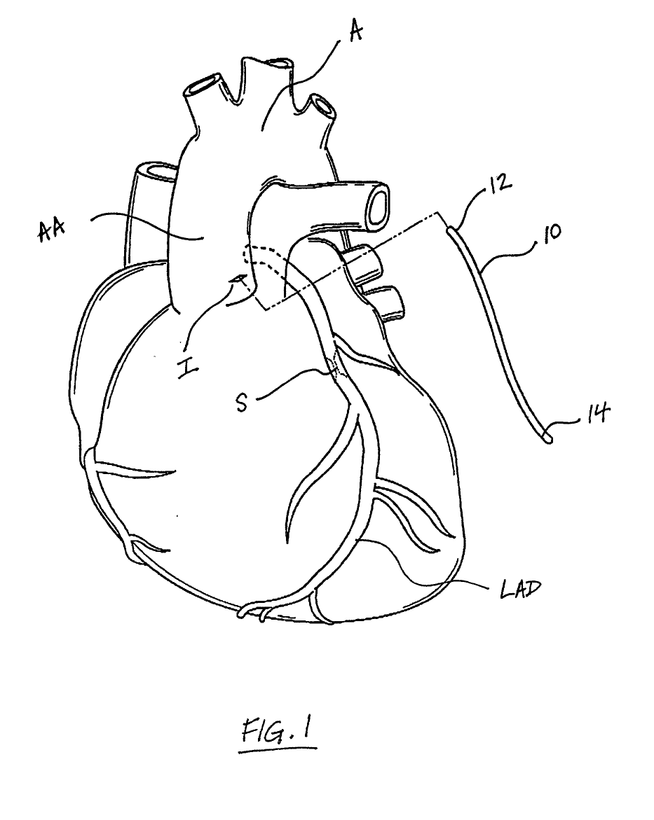

FIG. 1 shows the exterior of a patient's heart in perspective including the aortic arch A, the ascending aorta AA, and the left anterior descending artery LAD with a stenosis S in its lumen. A member 10 adapted to form a vascular structure that may be used as a graft is shown next to the heart. The member 10, which is preferably a mandrel with a solid or substantially solid outer surface, has ends 12, 14 which are used to place the formed graft in communication with two blood-carrying structures. In the illustrated embodiment these two structures are the ascending aorta AA and the LAD.

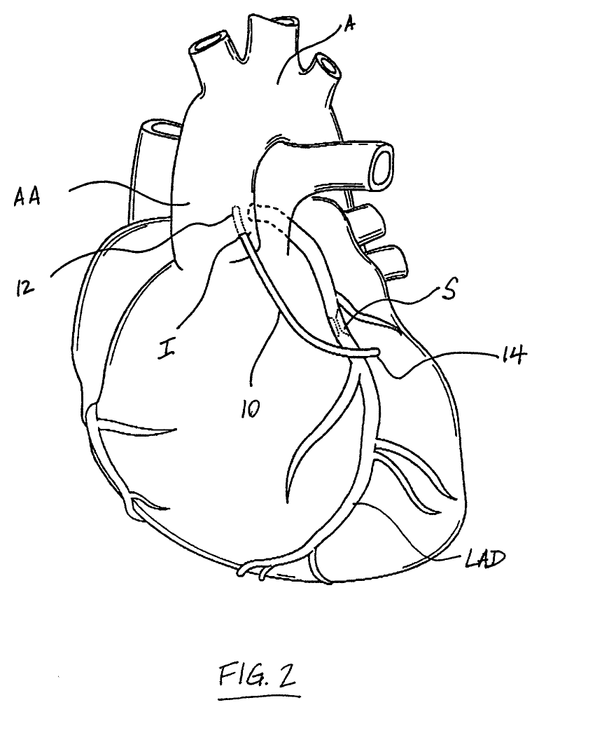

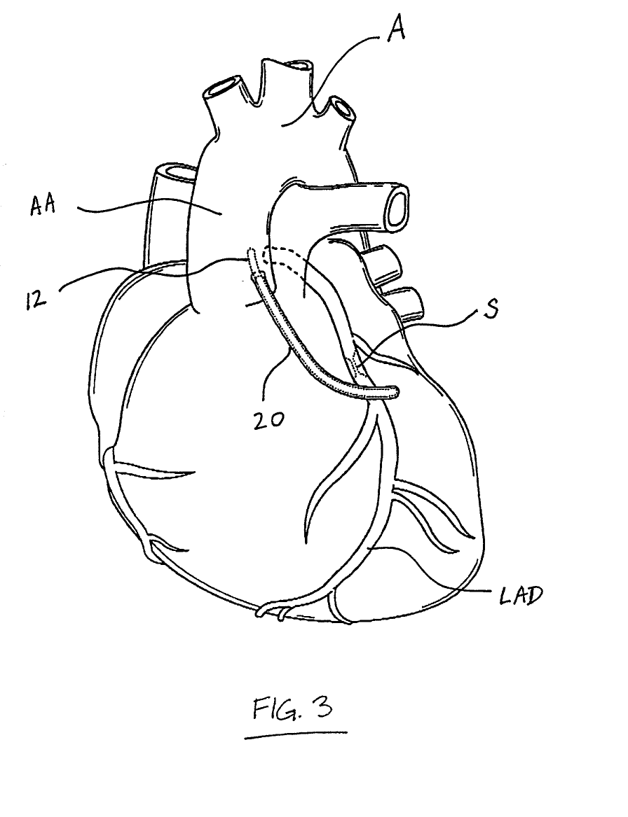

As seen in FIG. 1, the end 12 of the mandrel 10 is inserted into the lumen of the ascending aorta AA through an incision I formed in the aortic wall. FIG. 2 shows the mandrel end 12 in position while another portion of the mandrel 10 is located adjacent the LAD. This portion of the mandrel 10, located by the end 14 in this embodiment, is preferably positioned against the tissue of the arterial wall (distal to the stenosis S) in order to promote tissue growth around the mandrel. FIG. 3 schematically indicates a vascular structure 20 formed around the mandrel 10.

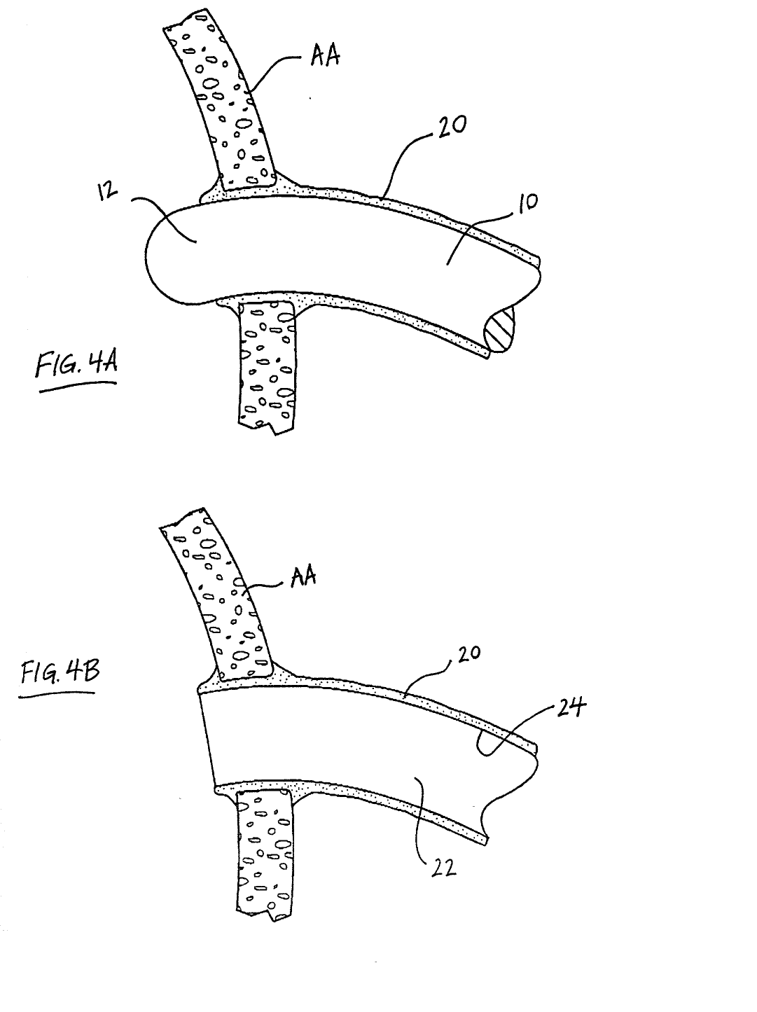

FIGS. 4A-4B show the proximal end of the vascular graft 20 that is formed by the mandrel end 12. FIG. 4A shows the end 12 of mandrel 10 as it is illustrated in FIG. 3, i.e., disposed in the ascending aorta AA with a mass of scar tissue around its exterior. FIG. 4B shows the proximal end of the vascular graft 20 without the mandrel 10. The mandrel 10 may, for example, be removed, absorbed, dissolved, disintegrated, etc. The resulting vascular structure 20 has a lumen 22 and a wall 24 and communicates with the lumen of the aorta.

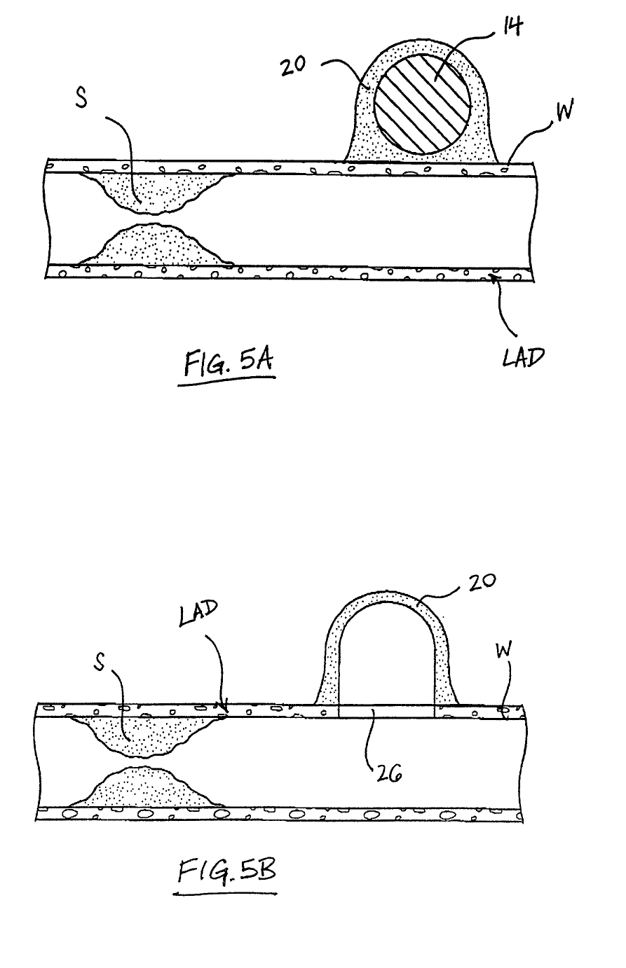

FIGS. 5A-5B show the LAD and the second end 14 of the mandrel 10 disposed against or just over the tissue of the vessel wall W. FIG. 5A shows comprehensive healing around the mandrel 10 with the aggregated cells encapsulating the mandrel to form a smooth continuous lining that define the lumen of the graft. FIG. 5B shows the vascular structure 20 without the mandrel 10, as well as an opening 26 through the LAD wall 24 (and through the newly-formed graft, if necessary) via which the graft and LAD communicate.

As can be seen from FIGS. 4A-4B and 5A-5B, the body has in effect grown or built a completely autologous graft conduit which may be used to bypass the stenosis S.

The opening 26 may be formed in any suitable manner, for example, by mechanical cutting or boring, applying laser or RF energy. Additionally, the opening may be supported and maintained open (or, alternatively, valved or otherwise controlled) by any suitable means, such as a stent or fabric sleeve.

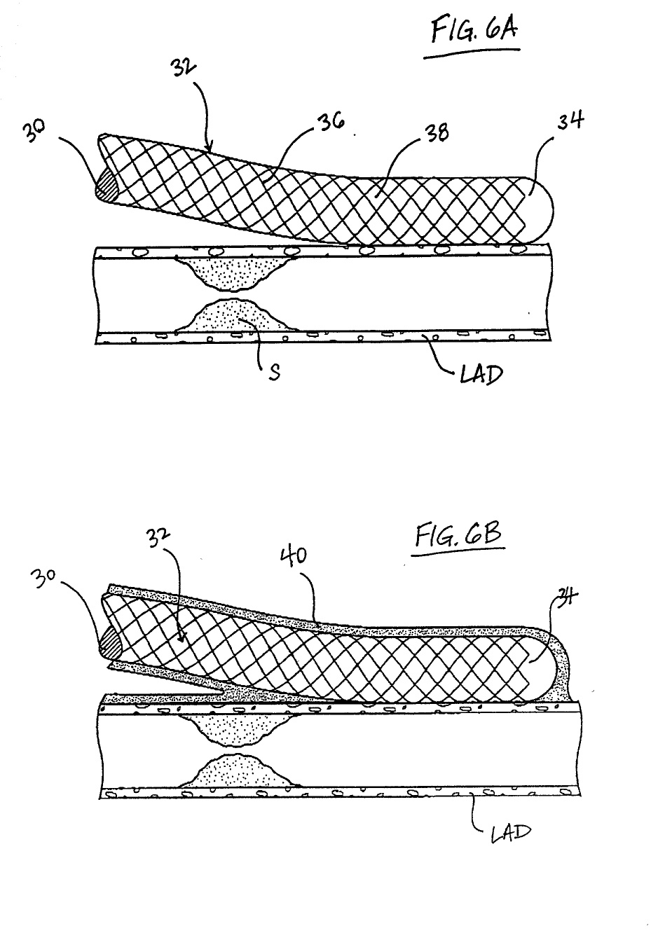

FIGS. 6A-6D are longitudinal sectional views of a mandrel 30 constructed according to another embodiment of the invention. The mandrel 30 is provided with a vessel support 32 adjacent an end 34 of the mandrel. The support 32 may take various forms but is primarily intended to act as an internal or external support for the wall of the vascular structure formed according to the invention, thereby maintaining the lumen of the structure open (or, in alternative embodiments, valve, meter, regulate or otherwise control flow through the graft). In FIG. 6A the support 32 comprises a wire frame 36 with openings 38 through which tissue may grow during the healing process.

FIG. 6B shows the mandrel 30 and vessel support 32 after a mass of scar tissue 40 has formed around their exterior. FIG. 6C shows the mandrel 30 being removed. One benefit of using a vessel support is to prevent damage from subsequent injury to the wall of the newly-formed vascular structure. This is exemplified by an aneurysm 42 shown in FIG. 6C. Without a vessel support there may be increased risk of the wall rupturing. The vessel support may therefore have a fabric or cloth liner to provide a solid layer if desired. FIG. 6D shows the completed creation of a graft vessel with an opening 44 formed in the wall of the LAD (or other target vessel) to communicate the lumens of the respective vessels. The vessel support 32 may have a window to communicate with the LAD, and it may be punched or otherwise opened before, during or after placement.

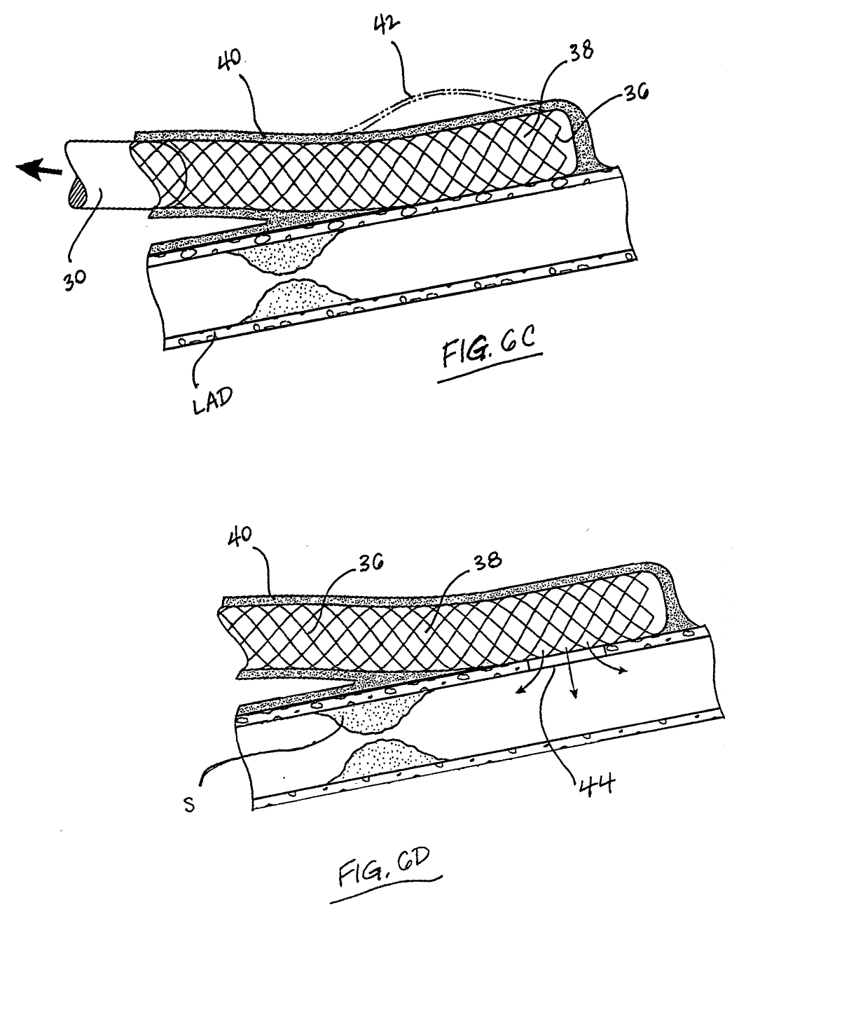

FIGS. 7A-7C show mandrels constructed according to other embodiments of the invention. FIG. 7A shows a mandrel 50 comprising a diamond stent pattern 52 over all or part of its length. FIG. 7B shows a mandrel 54 formed of a mesh which preferably may expand and contract. FIG. 7C shows a vessel support comprising a mandrel 56 with internal spacers 58 for centering the mandrel relative to the graft vessel created according to the invention. It will be recognized that the embodiments of FIGS. 7A-7C are only several of the many different configurations which this aspect of the invention could take, as the vessel support could be a stent, wire frame, wire coil, mesh, fabric, balloon, etc.

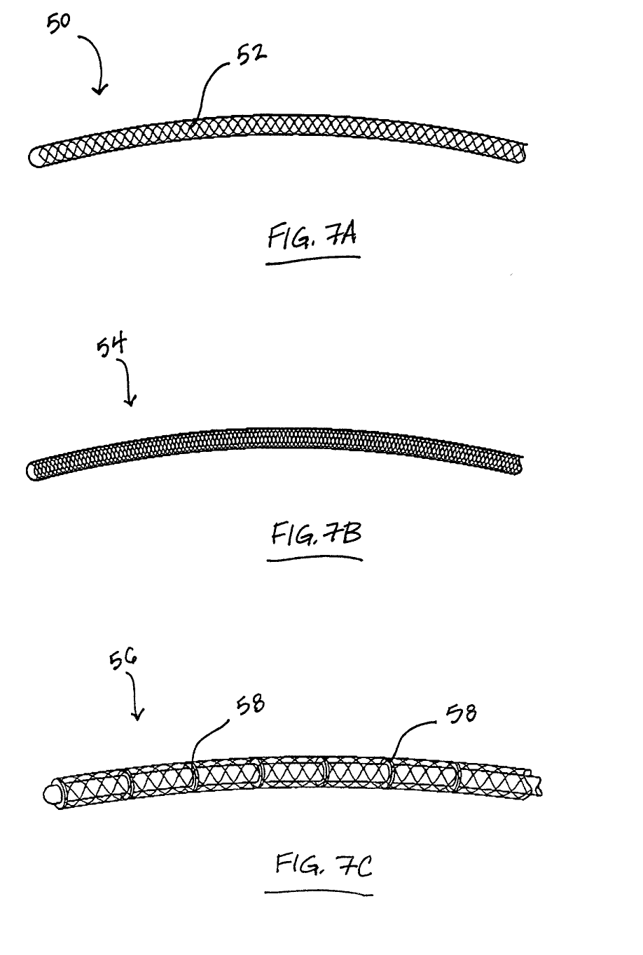

FIGS. 8-10 schematically show another embodiment of the invention wherein the vascular structure is created according to a percutaneous procedure, for example, in contrast to the procedure of FIGS. 1-3 which may be viewed as representing a surgical procedure. The invention also may be practiced in a minimally invasive (e.g., through small ports) manner

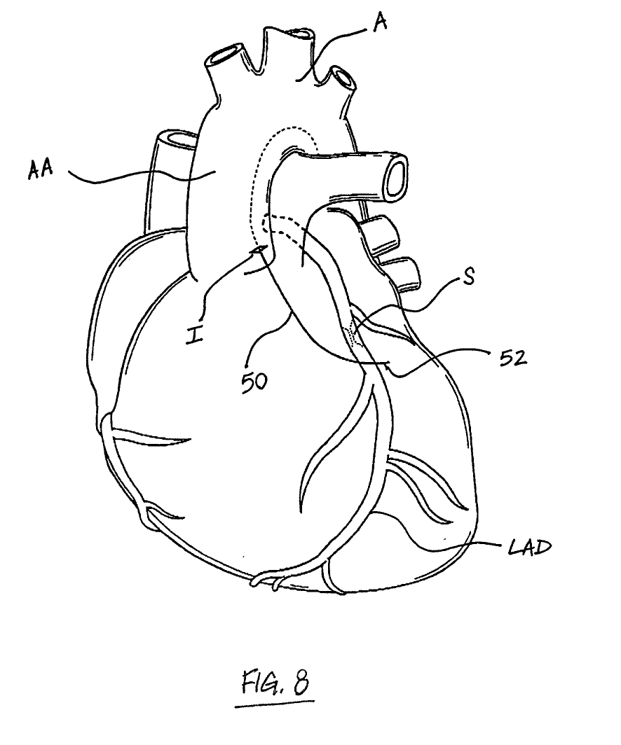

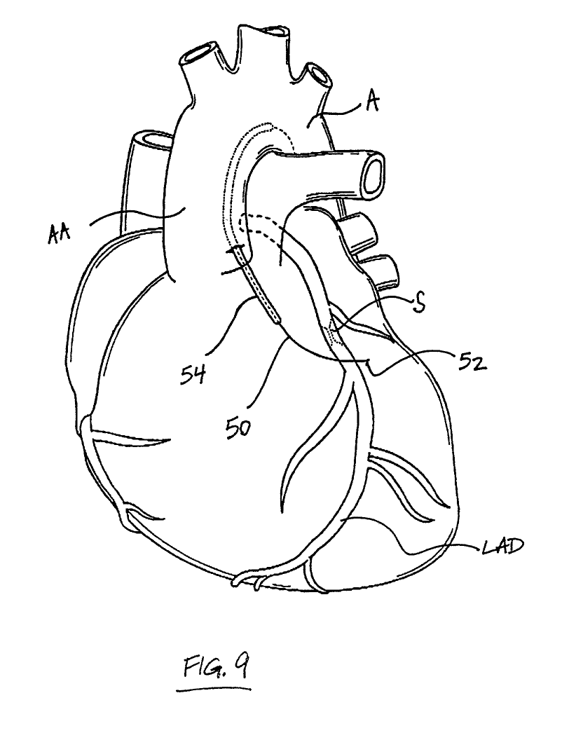

FIG. 8 shows a guide wire 50 placed in the patient's vasculature at a peripheral location (not shown) and guided past the aortic arch. An end 52 of the guide wire is passed out the incision I and is used as a rail to introduce one or more other devices. FIG. 9 shows a mandrel 54 with a bore (not shown) being guided along the wire 50, and FIG. 10 shows the mandrel 54 after it has been guided to its final position. At this point the guide wire 50 is removed and the formed graft 56 is placed in fluid communication with the LAD. The mandrel 54 is preferably sufficiently flexible to allow its delivery through a patient's vasculature.

The invention may be practiced percutaneously using guide catheters or cannulae, steering mechanisms, radiopaque marking, ultrasound guidance, or any other means for determining the position of a suitable delivery system, e.g., a sheath covering the vessel support.

The invention may also be practiced using mandrels having a mechanism for maintaining, and preferably fixing or substantially fixing, the mandrel in a desired position with respect to tissue at the site where the graft is being created. Exemplary mechanisms include mechanical tissue engaging elements such as barbs or hooks, as well as suture and adhesive.

The components of the invention may be formed of any suitable biocompatible materials, e.g., polymers including silicone or urethane, metals such as titanium, nitinol or stainless steel, or other materials possessing the desired properties. The mandrels also may be coated with a variety of substances for achieving various results. For example, any biological or pharmacological substance may be applied to, impregnated in, or carried by the mandrel.

Finally, it will be appreciated that the particular characteristics and properties of the mandrel, such as size, hollow or solid, degree of flexibility, ease of removal from the graft, etc., will be selected based on the specific application and user preference.

In one embodiment, the invention may be viewed as comprising a two-stage procedure. In the first stage, material is introduced (for example, percutaneously) which causes one ore more autologous vessels to form in the patient. In the second stage (for example, three to four weeks later) devices such as catheters and collapsible connectors may be delivered percutaneously and used to connect new vessel to the two target vessels. As mentioned above, internal or external bolsters, such as woven DACRON, may be used to prevent aneurysm formation and gene therapy may be used to refine the vascular growth process.

Five animal experiments were conducted with the goal of determining if it was possible to create a small diameter vascular conduit in a relatively short period of time that would support blood flow without leakage. We selected high flow conditions in order to subject the graft to a relatively strong test of hemostatic capability. Synthetic mandrels, formed of silicone and utilizing the same external surface chemistry and morphology, were implanted in five swine weighing between 40 and 50 Kg.

The mandrels were implanted between the aorta and right atrium. In each animal a mandrel was inserted within the pericardial sack and ran from the animal's aorta to the right atrium. In one of the animals, a second mandrel ran from the aorta to the right ventricle. Implants were done surgically on the beating heart through a thoracotomy incision. Mandrels were removed at approximately the three-week interval and blood flowed through the new conduit.

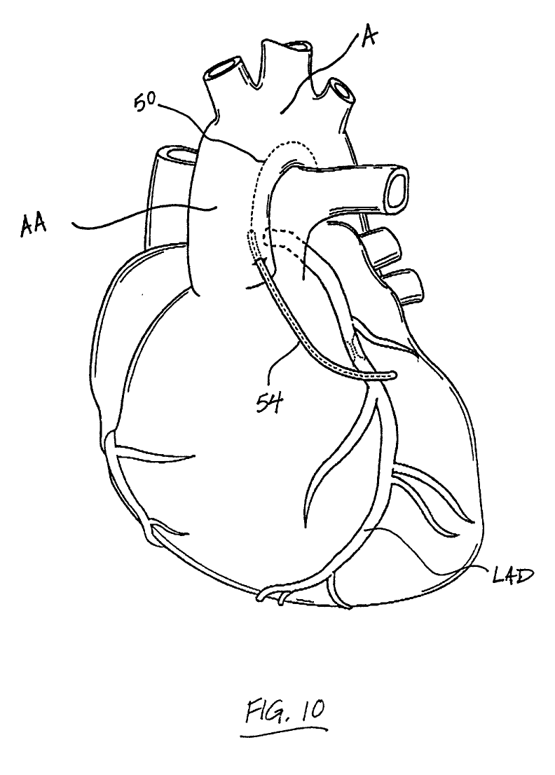

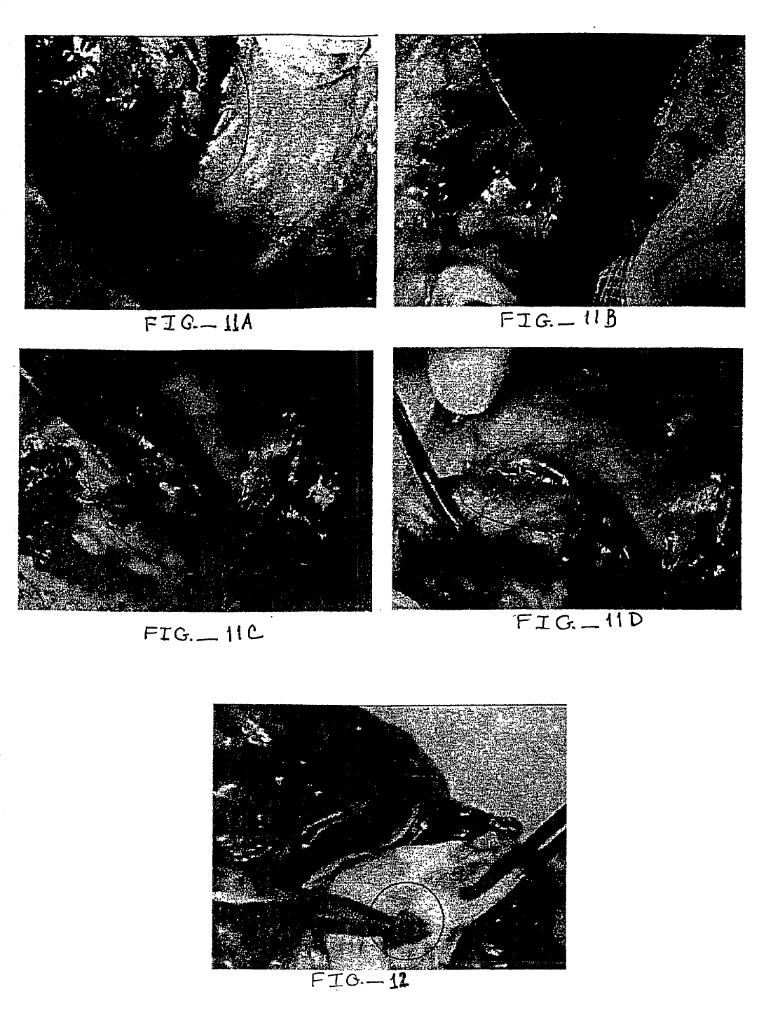

FIGS. 11A-11D are photographs of some of the vascular structures created during these studies. FIGS. 11B and 11C show a heart with one newly-formed graft (encircled), while FIGS. 11A and 11D show a heart with two newly-formed grafts (also encircled). FIG. 12 is a view of the inner surface of the aortic wall showing the end (encircled) of a newly-formed graft.

Angiograms taken one hour before sacrifice demonstrated excellent patency, despite the fact that experimental animals had not received either heparin or antiplatelet therapy at any point in the study. Three weeks after implantation, the mandrels were removed and each graft was evaluated. Both angiographic and surgical findings confirmed that the engineered vascular structure was patent and hemostatic. Histological data on the newly-formed graft demonstrated a highly vascular, fibrous capsule that appeared to be a natural vessel.

The invention thus provides the ability to “build” a completely autologous vessel, which has significant appeal for many applications, cardiothoracic being exemplary only. By using the body's own tissue, there may be less reactivity to the graft, thereby eliminating some of the patency failures that are routinely experienced with nonharvested grafts. This would allow formation of a graft that may be used without dissection from the patient's existing vascular system, yet produces equivalent or substantially equivalent patency as compared to autologous vessels.

The invention had been described primarily in connection with its preferred embodiment, namely, creating a vascular graft within the chest cavity for use as a bypass conduit during a CABG procedure. Some examples of other procedures in which the invention may be used are femoral-femoral, femoral-popliteal, femoral-tibial, iliofemoral, axillary-femoral, subclavian-femoral, aortic-bifemoral, aorto-iliac, aorto-profunda. femoris and extra-anatomic bypasses. It will be appreciated that these too are merely exemplary uses of the invention.

The preferred embodiments of the invention are described above in detail for the purpose of setting forth a complete disclosure and for sake of explanation and clarity. It will be readily understood that the scope of the invention defined by the appended claims will encompass numerous changes and modifications.

What is claimed is:

1. A method for forming in situ a vascular graft having a lumen adapted to place at least two blood vessels in fluid communication, the method comprising steps of:
(a) providing a member sized and configured to substantially define a lumen of a vascular graft;
(b) placing at least a portion of the member adjacent tissue located in a patient's body;
(c) substantially maintaining the portion of the member in a desired position to form a vascular graft with a lumen at the desired position; and
(d) using the vascular graft to place at least two blood vessels in fluid communication without substantially moving the graft from the desired position.

2. The method of claim 1, further comprising removing the portion of the member from the desired position.

3. The method of claim 2, wherein the member comprises a mandrel that breaks down over time.

4. The method of claim 1, wherein step (c) is carried out for a sufficient time to allow tissue healing around an exterior of the member, and the tissue healing creates the vascular graft while the exterior of the member defines the lumen of the vascular graft.

5. The method of claim 1, further comprising the step of placing the member at least partially within blood flow.

6. The method of claim 1, further comprising the step of providing the member with a support adapted to maintain the lumen vascular graft open.

7. The method of claim 1, further comprising providing the member with a substance adapted to be delivered to tissue adjacent the member.

8. The method of claim 1, wherein the at least one of steps (b), (c) and (d) is performed percutaneously.

9. A method for forming a vascular structure in situ at a location in a patient's chest or thoracic cavity, the vascular structure having a lumen and the method comprising steps of:
(a) providing a member adapted to define at least in part a vascular structure;
(b) placing at least a portion of the member in a desired position adjacent tissue located in a patient's chest or thoracic cavity;
(c) substantially maintaining the portion of the member in the desired position within the chest or thoracic cavity to form a vascular structure with a lumen at the desired position; and
(d) using the vascular structure to deliver blood.

10. The method of claim 9, wherein the location is within the pericardial space, the tissue is myocardial tissue and the vascular structure comprises a bypass graft disposed adjacent the exterior of the patient's heart, and step (d) is performed by using the graft to deliver blood from a source to a coronary vessel.

11. The method of claim 10, wherein at least step (b) is carried out surgically, and step (c) is performed by anchoring the portion of the member in the desired position.

12. The method of claim 9, wherein step (c) is performed with the member at least partially exposed to blood flow, and the member has an outer diameter in the range of from about 3 mm to about 5 mm.

13. The method of claim 12, wherein an end of the member is placed in the aorta to form a proximal end of the graft.

14. A system for forming a vascular structure having a lumen, the system comprising:
a mandrel having an exterior surface sized and configured to form a vascular structure as a result of tissue healing around the surface of the mandrel;
a positioning mechanism allowing the mandrel to be placed in a desired position relative to tissue so that a vascular structure may form at the position; and
wherein the mandrel is configured to allow the vascular structure to be used as a graft without harvesting or removing the vascular structure after formation.

15. The system of claim 14, wherein the mandrel comprises a material that allows removal of the mandrel after the vascular structure has formed to a desired degree.

16. The system of claim 14, wherein the mandrel is sufficiently flexible to allow its delivery through a patient's vasculature.

17. The system of claim 14, wherein the positioning mechanism is selected from the group consisting of the mandrel having a self-retaining configuration, mechanical tissue-engaging elements, suture and adhesive.

18. The system of claim 14, wherein the mandrel has a bore to allow delivery over a guide member.

19. The system of claim 14, further comprising a vessel support provided on the mandrel, the vessel support being selected from the group consisting of stents, wire frames, wire coils, meshes, fabrics and balloons.

20. The system of claim 14, further comprising a biological or pharmacological substance carried by the mandrel.
